(19) United States
(12) Patent Application Publication
(10) Pub. No.: US 2023/0220460 A1
(43) Pub. Date: Jul. 13, 2023

(54) Nucleic acid-based membrane constructs for RNA polymerase detection

(71) Applicant: University of Seoul Industry Cooperation Foundation, Seoul (KR)

(72) Inventors: Jong Bum Lee, Seoul (KR); Da Jeong Kim, Seoul (KR); Sang Woo Han, Seongnam-si (KR)

(21) Appl. No.: 18/079,262

(22) Filed: Dec. 12, 2022

(30) Foreign Application Priority Data
Dec. 10, 2021 (KR) 10-2021-0176340

(51) Int. Cl. C12Q 1/686; C12Q 1/6825; C12N 9/12; C12Q 1/6888

(52) U.S. Cl. CPC C12Q 1/686; C12Q 1/6825; C12N 9/127; C12Q 1/6888

(57) ABSTRACT

Provided is a kit for virus detection including a nucleic acid membrane containing a gold component and reacting with RNA polymerase to transcribe RNA, a biosensor for RNA polymerase detection based thereon, and RNA polymerase.

### CROSS-REFERENCE TO RELATED APPLICATION

This application claims priority to Korean Patent Application No. 10-2021-0176340, filed on Dec. 10, 2021.

### REFERENCE TO AN ELECTRONIC SEQUENCE LISTING

The contents of the electronic sequence listing (2023-03-14_Sequence_Listing.xml; Size: (13,028 bytes; and Date of Creation: Mar. 14, 2023) is herein incorporated by reference in its entirety.

### BACKGROUND

### Field

The present disclosure relates to a nucleic acid-based membrane construct for RNA polymerase detection and a biosensor for detecting general-purpose viruses based thereon. The present disclosure was supported by a project from the Ministry of Science and ICT (MSIT), the National Research Foundation of Korea (NRF) supported by the Korean government, (Project Information: NRF-2022R1A2C2004820).

### Related Art

The COVID-19 pandemic has made us aware of the importance of rapid and accurate diagnosis of viral infections. Currently, RT-qPCR-based analysis methods are mainly used to diagnose viral infections. In addition, point-of-care testing (POCT) based on serological and immunological analysis methods for detecting specific antigens or antibodies in saliva or serum is widely used. However, these detection methods require target-specific designs and take time to develop, limiting rapid response in the early stages of the pandemic. In addition, target-specific diagnostic kits require revalidation to determine the ability to detect newly emerged mutations. Therefore, there is a need for analysis methods independent of viral species and mutations that do not require a revalidation process for newly emerged viruses.

On the other hand, RdRP (RNA-dependent RNA polymerase) is a type of replicase-transcriptase complex, which is an essential protein for RNA replication and transcription of viruses such as Ebola virus, Zika virus, hepatitis C virus (HCV), and coronavirus. RdRP has no sequence homology between viruses but is common in terms of RNA replication function. RdRP uses backtracked reversal replication capability to rapidly amplify RNA. Thus, RdRP can be a commonly applicable biomarker for various RNA viral infections. However, there is no known universal biosensor that can detect various types of viruses by targeting RdRP.

(Patent Document 1) Korean Publication No. 10-2015-0057247 (May 28, 2015)

### SUMMARY

The present disclosure relates to a nucleic acid-based membrane construct for RNA polymerase detection. In an aspect, the present disclosure provides a plurality of nucleic acid strands partially complementary; and a gold component bound to the nucleic acid strand; wherein the nucleic acid strand provides an RNA polymerase-activated nucleic acid membrane including an exposed end that reacts with RNA polymerase.

The RNA polymerase-activated nucleic acid membrane can transcribe RNA strands regardless of the type of RNA polymerase. RNA strands transferred to the surface of the nucleic acid membrane can block the access of materials that can react with the gold component. Therefore, when used with substances that can react with gold components and cause specific reactions such as color change, it can be used to detect whether RNA polymerase is included in the sample, and furthermore, it can be used to universally detect viruses containing RNA polymerase.

The present disclosure has an effect that is difficult to predict based on the prior art in that the nucleic acid is based on a scaled-up membrane, it is confirmed that the gold-metalized nucleic acid membrane can have sufficient physical properties and stability to be used as a biosensor, it is confirmed that the nucleic acid-based membrane subjected to metallization and reduction can transcribe RNA by RNA polymerase, the access of a material that reacts with gold is blocked by the transcribed RNA as a detection principle, and any virus having an RNA polymerase that is not specific to the type of virus can be detected.

According to one embodiment, the nucleic acid may be DNA or RNA.

The sequence of the nucleic acid strand is not particularly limited as long as it can react with RNA polymerase to cause RNA transcription and block the access of samples that can react with gold by shielding the surface of the nucleic acid membrane.

The nucleic acid strand may be a rolling circle amplified (cRCA) DNA or a rolling circle transcription (cRCT) RNA from two types of circular DNA that are partially complementary to each other.

According to one embodiment, the RNA polymerase may be a T7 RNA polymerase or an RNA-dependent RNA polymerase (RdRP).

According to one embodiment, the gold component may be a gold ion, gold nanoparticles, or a combination thereof. According to one embodiment, the diameter of the gold nanoparticles may be 1 to 200 nm, 10 to 150 nm, or 50 to 100 nm.

According to one embodiment, the nucleic acid membrane may be formed a structure (RNA bump) composed of RNA transcribed on the surface when reacting with RNA polymerase. The RNA bump may block the material having a redox reaction with the gold component from accessing the gold component.

Another aspect provides a biosensor for RNA polymerase detection including the RNA polymerase-activated nucleic acid membrane.

Another aspect provides a kit for virus detection including an RNA polymerase including a dye that changes color by causing the biosensor and gold to redox reaction.

The dye that changes color by causing the redox reaction with the gold may be TMB (3,3′,5,5′-tetramethylbenzidine). TMB can be oxidized by gold to produce oxidized TMB (oxTMB) showing blue color.

The virus including the RNA polymerase may be a virus including positive-stranded RNA, a virus including double-stranded RNA, and a virus including negative-stranded RNA. For example, the dsRNA virus may be Birnaviridae, Chrysoviridae, Cystoviridae, Endornaviridae, Hypoviridae, Megabirnaviridae, Partitiviridae, Picobirnaviridae, Reoviridae, or Totiviridae. The benign-stranded ssRNA virus includes Nidovirales (Arteriviridae, Coronaviridae, and Roniviridae), Picornavirales (Dicistroviridae, Iflaviridae, Marnaviridae, Picornaviridae, Secoviridae, Bacillariornavirus, and Labyrnavirus), and Tymovirales (Alphaflexiviridae, Betaflexiviridae, Gammaflexiviridae, and Tymoviridae); as an unassigned family, Alphatetraviridae, Alvernaviridae, Astroviridae, Barnaviridae, Bromoviridae, Caliciviridae, Carmotetraviridae, Closteroviridae, Flaviviridae, Leviviridae, Luteoviridae, Narnaviridae, Nodaviridae, Permutotetraviridae, Potyviridae, Togaviridae, Tombusviridae, and Virgaviridae; as an unassigned genus, Benyvirus, Cilevirus, Hepevirus, Idaeovirus, Ourmiavirus, Polemovirus, Sobemovirus, and Umbravirus; and as an unassigned species, Botrytis virus F, Canine picodicistrovirus, Chronic bee paralysis associated satellite virus, Heterocapsa circularisquama RNA virus, Le Blanc virus, Orsay virus, and Santeuil virus. The negative-stranded ssRNA virus may be Mononegavirales (Bornaviridae, Filoviridae, Paramyxoviridae, and Rhabdoviridae); as an unassigned family, Arenaviridae, Bunyaviridae, Ophioviridae, and Orthomyxoviridae; and as an unassigned genus, Deltavirus, Emaravirus, Nyavirus, Tenuivirus, Orchid fleck virus, and Taastrup virus.

Another aspect provides a method for preparing an RNA polymerase-operated nucleic acid membrane including preparing a nucleic acid membrane; mixing the nucleic acid membrane and gold ions to prepare a nucleic acid membrane to which gold ions are bound; and reducing the nucleic acid membrane to which the gold ion is bound.

The step of preparing the nucleic acid membrane is performed by complementary rolling circle amplification (cRCA) of DNA from two types of circular DNA partially complementary to each other, or by complementary rolling circle transcription (cRCT) of RNA, and by evaporative oil ceramics assembly to prepare a nucleic acid membrane in which the nucleic acid strands are highly entangled and concentrated.

The step of mixing the nucleic acid membrane and the gold ion may be mixing and incubating HAuCl₄ with the prepared nucleic acid membrane.

The step of reducing the nucleic acid membrane to which the gold ions are bound may be reacting with hydroxyamine hydrochloride (HAHC). Gold ions bound to nucleic acid are converted into gold nanoparticles by the step of reducing, and the ends of the nucleic acid strands that can be recognized by RNA polymerase can be exposed.

Another aspect provides an RNA polymerase-containing virus detection method includes contacting a sample to confirm whether a virus comprising an RNA polymerase-activated nucleic acid membrane and an RNA polymerase is present; contacting a dye that changes color by a redox reaction when in contact with the RNA polymerase-operated nucleic acid membrane and gold in contact with the sample; and determining that the sample contains RNA polymerase if the color does not change.

According to one embodiment, since the RNA polymerase-activated nucleic acid membrane targets RNA polymerase, any virus containing RNA polymerase can be detected regardless of type.

### BRIEF DESCRIPTION OF THE DRAWINGS

FIGS. 1A and 1B schematically show the production process of RANAM and a RANAM biosensor-mediated RNA polymerase detection process.

FIG. 1A shows a process of manufacturing a NA membrane by complementary rolling circle replication (cPCR) using two complementary circular DNA strands A and B. The DNA membrane was produced through complementary rolling circle amplification (cRCA) using two partially complementary circular DNA strands and phi29 DNA polymerase. RNA membranes were fabricated by circular DNA and complementary rolling circle transcription (cRCT) containing T7 promoter regions. The cRCR solution was concentrated by EISA and the NA strands were entangled and self-assembled into the NA membrane.

FIG. 1B shows a process of fabricating an Au-NA membrane completely covered with gold ions. The subsequent reduction allows partial exposure of the NA strand, enabling recognition by RNA polymerase.

FIG. 2 shows the characteristics of NA membranes, Au-NA membranes, and RANAM. FIG. 2 shows an image of a DNA membrane, an Au-DNA membrane, and D-RANAM, and a TEM image. The Au-DNA membrane was successfully metallized. D-RANAM has grown internal AuNP and has been reduced to dark green. The inserted tick bar represents 10 nm.

FIG. 3A shows a non-contact surface profiler image and roughness and FIG. 3B shows a FE-SEM image of a DNA membrane and an Au-DNA membrane. The size of the non-contact surface profiler image is 130 μm×95 μm.

FIG. 4 shows a production process and digital image of a yellow Au-RNA membrane and R-RANAM.

FIG. 5 is a result of confirming the mechanical properties of the dehydrated Au-RNA membrane and R-RANAM.

FIG. 6A shows a color change to blue as oxTMB is formed by redox reaction between the Au-DNA membrane and D-RANAM with TMB. The reaction is the result of proceeding for 90 minutes. The scale bar represents 2 mm.

FIG. 6B shows a process in which an Au-DNA membrane reacts with TMB for 1 hour and changes color. The time lapse is 0 minutes, 5 minutes, 15 minutes, 30 minutes, and 60 minutes. The RGB image was converted to an HSB image, and the hue value was quantified.

FIG. 7 shows a change in the absorbance spectrum due to the reaction of the Au-DNA membrane and TMB. After reaction with TMB, strong absorbance was observed at oxTMB characteristic wavelength of 670 nm.

FIG. 8 shows the result of TMB-mediated redox reaction in the presence of T7 RNA polymerase at different concentrations of D-RANAM. The newly generated RNA transcripts were inhibited in redox reactions as the concentration of T7 RNA polymerase increased.

FIG. 9 shows an RdRP detection platform based on TMB redox reaction inhibition by RdRP-mediated RNA amplification.

FIG. 10 shows a digital image showing that the TMB redox reaction is inhibited according to the RdRP transcription time of R-RANAM.

FIG. 11A and FIG. 11B show the results of confirming by absorbance spectrum that the TMB redox reaction is inhibited according to the RdRP transcription time of R-RANAM.

FIG. 12A and FIG. 12B show the results of confirming that SYBR green I stained R-RANAM fluorescence varies with or without RdRP.

FIG. 13 shows that R-RANAM forms an RNA bump on the surface by RdRP. The tick bar represents 500 nm.

FIG. 14 shows the results confirming that R-RANAM can inhibit redox reactions by TMB by very low RdRP concentration.

FIG. 15A and FIG. 15B show the results of FIG. 14 in an absorbance spectrum.

FIG. 15C shows a fluorescence image by SYBR green I stain of R-RANAM (RdRP-) and R-RANAM (RdRP+) treated with 100 pM RdRP.

FIG. 16 shows a smartphone-assisted prototype kit assembled using a 3D printed scaffold and R-RANAM.

FIG. 17A and FIG. 17B show the result of applying test samples of RdRP− and RdRP+ to the prototype kit of FIG. 16. The grid bar represents 5 mm.

FIG. 18 shows the result of confirming the change in gray value after storing the prototype kit at room temperature for 20 days.

### DESCRIPTION OF EXEMPLARY EMBODIMENTS

Hereinafter, one or more embodiments will be described in more detail through examples. However, these embodiments are intended to illustrate one or more embodiments illustratively, and the scope of the present invention is not limited to these embodiments.

Experimental Method

1. Circular DNA Production

Linear DNA was produced by mixing 92 nt or 87 nt phosphorylated DNA (IDT) with 22 nt primer DNA (IDT) in nuclease-free water. A mixed solution of linear DNA (10 μM) and primer (10 μM) was tempered while gradually cooling in a thermal cycle (Bio-Rad) at 95° C. for 5 minutes and at 25° C. for 1 hour to produce circular DNA. The 99nt, 87nt, and 22nt DNA used to make circular DNA are shown in Tables 1 and 2 below.

TABLE 1

| Cir X | DNA strands (nt) | Sequences (5′-3′) |
|---|---|---|
| Cir 1 | Pri 1 for | AAC ATA ATG TCA CTA TAG GGA T |
| | cir 1 (22 nt) | |
| | Lin 1 for | /Phosphate/ ′ATA GTG ACA TTA TGT TGA TGG TAA′ |
| | cir 1(92 nt) | GTC ACC CCA ACC TGC CCT ACC ACG GAC ′′TCT |
| | | CTA TGT TGA TGG TAA TCG CTA TCT AGA GGC |
| | | ATA TCC CT′′ |
| Cir 2 | Pri 2 for | CTA GAG GCA TAT CCC TAT AGT G |
| | cir 2 (22 nt) | |
| | Lin 2 for | /Phosphate/ ′′AGG GAT ATG CCT CTA GAT AGC GAT |
| | cir 2 (92 nt) | TAC CAT CAA CAT AGA GA′′A ACC AAC CAC ACC |
| | | AAC CAA AGA AAT GA′T TAC CAT CAA CAT AAT |
| | | GTC ACT AT′ |
| Cir 3 | Pri 3 for | TAA TAC GAC TCA CTA TAG GGA T |
| | cir 3 (22 nt) | |
| | Lin 3 for | /Phosphate/ ′′′ATA GTG AGT CGT ATT AA′′′A AAC |
| | cir 3 (92 nt) | TTC AGG GTC AGC TTG CTT GC′′′T TAA TAC GAC |
| | | TCA CTA T′′′AG CGC AAA ACT TCA GGG TCA GCT |
| | | TGC TTA TCC CT |

Table 1 is a primer sequence for producing a DNA membrane, an Au-DNA membrane, and a D-RANAM. Pri X and Lin X were used to synthesize cir x (x is 1, 2, or 3). The complementary sequences are marked ‘, “, and”’. DNA membranes, Au-DNA membranes, and D-RNAM were synthesized using cir 1 and cir 2 as templates. The D-RANAM biosensor for T7 RNA polymerase encoded a T7 promoter and was fabricated using cir 3 with a magnetic complementary sequence.

TABLE 2

| | DNA strands | Sequences |
|---|---|---|
| Cir Y | (nt) | (5′-3′) |
| Cir 4, 5, | Pri 4 for | TAA TAC GAC TCA CTA TAG GGA T |
| 6, 7 | cir 4, 5, | |
| | 6, 7 | |
| | (22 nt) | |
| Cir 4 | Lin 4 for | /Phosphate/ ATA GTG AGT CGT ATT A′GG TCA CGA |
| | cir 4 | GGG TGG GCC AGG GCA CGG GCA GCT TGC CGG |
| | (92 nt) | TGG TGC AGA TGA ACT TCA GGG TCA GCT TGC |
| | | CG′A TCC CT |
| Cir 5 | Lin 5 for | /Phosphate/ ATA GTG AGT CGT ATT A′CG GCA AGC |
| | cir 5 | TGA CCC TGA AGT TCA TCT GCA CCA CCG GCA |
| | (92 nt) | AGC TGC CCG TGC CCT GGC CCA CCC TCG TGA |
| | | CC′A TCC CT |
| Cir 6 | Lin 6 for | /Phosphate/ ATA GTG AGT CGT ATT A′′AA TAA GGC |
| | cir 6 | TAT GAA GAG ATA CTT′′ GTA TCT CTT CAT AGC CTT |
| | (87 nt) | A′′′AA TAA GGC TAT GAA GAG ATA CTT′′′ ATC CCT |
| Cir 7 | Lin 7 for | /Phosphate/ ATA GTG AGT CGT ATT A′′′AA GTA TCT |
| | cir 7 | CTT CAT AGC CTT ATT′′′GTA TCT CTT CAT AGC CTT |
| | (87 nt) | A′′AA GTA TCT CTT CAT AGC CTT ATT′′ ATC CCT |

Table 2 is a primer sequence for fabrication of RNA membranes, Au-RNA membranes and RRANAM. Pri 4 and Lin Y were used to synthesize cir Y (Y is 4, 5, 6 or 7). Complementary sequences are denoted as ‘, “, and’”. RNA membranes, Au-RNA membranes and R-RNAM for analysis on RdRP time-dependent staining results and fluorescence and SEM images were synthesized with cir 4 and cir 5. In addition, R-RANAM biosensor and prototype kit assemblies for RdRP concentration-dependent detection experiments were synthesized with cir 6 and cir 7.

After producing circular DNA, the reaction solution was mixed with 1X ligation buffer (30 mM Tris-HCl, 10 mM MgCl₂, 10 mM DTT and 1 mM adenosine triphosphate) and T4 DNA ligase (0.03 U m1⁻¹, Promega), and incubated at room temperature overnight to ligate Nick of circular DNA.

To purify circular DNA, zeba spin desalting column (Thermo Scientific) was used, and Exonuclease I & III in 2U and 10U was treated at 37° C. to remove non-circular DNA. The solution was then incubated at 80° C. for 30 minutes to deactivate Exonuclease. Purified circular DNA (1 μg) was analyzed with 3% agarose gel electrophoresis at 80 mV for 110 minutes, stained with GelRed, and then imaged with Gel Doc EZ imager (Bio-Rad).

2. Nucleic Acid (NA) Membrane Preparation by Complementary Rolling Circle Replication (cPCR) and Evaporative Oil Ceramics Assembly (EISA) of Two Circular DNA

(1) DNA Membrane Manufacturing

Complementary rolling circle amplification (cRCA) was performed with two circular DNA fragments partially complementary to each other to synthesize the DNA membrane. An equimolar solution containing primer and circular DNA was incubated at room temperature for 2 hours to hybridize. Two circular DNA fragments were mixed with a final concentration of 0.5 μM, a 2 mM deoxyribonucleotide triphosphate mix, 1X phi 29 DNA polymerase buffer (50 mM Tris-HCl, 10 mM (NH₄)₂SO₄, 4 mM dithiothreitol, 10 mM MgCl₂, pH 7.5) and 1U μl⁻¹ phi 29 DNA polymerase. The reaction mixture was incubated at 30° C. for 4 hours to perform cRCA, and the tube was opened and evaporated overnight at 30° C.

(2) RNA Membrane Manufacturing

Two circular DNA fragments containing the T7 promoter region and partially complementary were used to synthesize RNA membranes. Two circular DNA fragments were incubated at 37° C. for 20 hours with a final concentration of 3 μM, a 3.75 mM ribonucleotide triphosphate mix, 2X reaction buffer (60 mM Tris-HCl, 9 mM MgCl₂, 1.5 mM DTT and 3 mM spermidine, pH 7.9), and T7 RNA polymerase (NEB) in 5U μl⁻¹ to perform complementary rolling circle transcription (cRCT). After opening the tube to allow EISA, the reaction mixture was evaporated overnight at 37° C. After the EISA process, the remaining reactants were removed and washed 4 times with nuclease-free water.

3. Synthesis of RNAM (RNA Polymerization Actuating Nucleic Acid Membrane) by Metallization of Nucleic Acid (NA) Membrane

3-1. Au-NA Production

To prepare the metallized NA membrane, a nucleic acid (NA) membrane and 2 mM of HAuCl₄ were mixed and incubated overnight at room temperature. Excess Au³⁺ was removed by washing three times with nuclease-free water.

3-2. RANAM Production

5 mM of hydroxylamine hydrochloride (HAHC, Alfa Aesar) was added to the Au-NA membrane to de-shield the membrane surface and expose the NA strands. After incubation at room temperature for 30 minutes, the resulting RNAM was thoroughly washed with nuclease-free water and stored at 4° C. until use.

4. Characterization of NA membranes, Au-NA membranes, and RANAMs

A bright-field microscope (Korea Lab Tech Corporation, KI-400) was used to observe colorimetric changes in the membrane. Transmission electron microscopy (TEM; JEOL, JEM2100F) and scanning electron microscopy (STEM) were used to investigate the internal structure and gold nanoseed generation. Field-Emission scanning electron microscope (FE-SEM; Hitachi, SU8010) to obtain high-resolution images.

The roughness of the membrane surface was observed using a non-contact surface profiler (Bruker, Contour GT-K), and the roughness was expressed as the root mean square (Rq) of the profile.

In addition, the surface and morphological changes of the membrane were visualized using an atomic force microscope (AFM; Park Systems, NX10). The absorbance of NA membranes, Au-NA membranes, and RANAMs was measured using a spectrophotometer (Thermo Scientific, Nanodrop 2000c).

5. RNA Transcription Activity by DNA-Based RANAM (D-RANAM) and T7 RNA Polymerase

Cir 3 (Table 1), including a T7 promoter, was used to fabricate and metallize the DNA membrane to produce D-RANAM. And then, a single DRANAM was then incubated at 37° C. for 2 hours with a 1 mM ribonucleotide solution mix, 2X RNA polymerase reaction buffer (80 mM Tris-HCl, 12 mM MgCl₂, 2 mM DTT, 4 mM spermidine), 0.8U μl⁻¹ RNase inhibitor, and T7 RNA polymerase (0, 10 or 40U μl⁻¹) to detect T7 polymerase activity of D-RANAM. The colorimetric changes of D-RANAM were observed using a bright-field microscope.

6. Production of Recombinant RdRP Protein from E. coli

RdRP (RNA dependent RNA polymerase) protein was expressed and purified from transgenic E. coli. As a first step, the bacteriophage Φ6 RdRP gene (NC_003715.1) was PCR-amplified using an RdRP DNA fragment synthesized with primers RdRP-F (5′-cttacatatgccgaggagagcccacgag-3′) and RdRPR RdRPR (5′-gactctcgagcctcggcattacagaacgga-3′) as a template.

The amplified PCR products were decomposed into NdeI and XhoI and ligated with NdeI/XhoI degraded pET22b plasmid (Novagen, USA). E. coli BL-21 (DE3) was transformed with the resulting plasmid. RdRP protein-expressing E. coli BL-21 (DE3) strain was incubated at 37° C. in Luria-Bertani (LB) medium. Incubated E. coli was treated with 0.2 mM IPTG and incubated at 16° C. for 20 hours to induce RdRP expression. The cultured strains were centrifuged at 3134×g for 20 minutes to collect cell pellets. The cell pellets were resuspended in a 20 ml buffer A solution (50 mM sodium phosphate, 300 mM NaCl, 20 mM β-mercaptoethanol, 0.5 mM PMSF, 10% (v/v) glycerol, pH 7.5) containing 10 mM imidazole. The cell pellets were sonicated with Vibra-Cell VCX 130 (Sonics & Materials Inc., USA) to be dissolved, and supernatants were collected and loaded into Ni²⁺-NTA columns (Invitrogen, USA, R901-15). The RdRP protein bound to the Ni²⁺-NTA column was eluted into buffer A solution containing 300 mM imidazole. The eluted solution was desalted by dialysis with buffer B (50 mM HEPES, 100 mM NaCl, 20 mM β-mercaptoethanol, pH 7.5) overnight at 4° C. The dialyzed RdRP protein was concentrated in a centrifugal filter unit (30 kDa MWCO, Millipore) and stored supplemented with 20% (v/v) glycerol. Protein concentrations were determined using the Bradford assay with bovine serum albumin as the standard.

7. RNA-Directed RNA Transcription of RNAM by RdRP

RANAM was incubated with a 1 mM ribonucleotide solution mix, 40 mM Tris-HCl, 0.5 mM MgCl₂, 2 mM MnCl₂, 10 mM ammonium acetate, 0.8U μl⁻¹ Ribonuclease inhibitor (Promega, RNasin®), and RdRP (100 aM, 100 fM, 100 pM, or 100 nM) at 30° C. for 2 hours. After RNA transcription, RANAM was stained with SYBR green I to quantify RNA-directed RNA transcription by RANAM, and fluorescence images were taken using inverted fluorescence microscope (Nikon, Eclipse TiU). To investigate the surface of RANAM, samples were prepared on Si wafers and observed using FE-SEM.

8. Redox Reaction and Colorimetric Signal Derivation Using TMB

RANAM transferred with RdRP was washed with nuclease-free water to remove excess reactants. The dehydrated RANAM was then incubated with TMB solution (3,3′,5,5′-tetramethylbenzidine) at room temperature for 5 minutes. The colorimetric change of RANAM was observed macroscopically using a digital camera or using a bright-field microscope. A spectrophotometer was used to measure the absorbance spectrum.

9. Raman Spectrum Analysis of RNA Membranes and R-RANAM

R-RANAM transferred with RNA membrane, R-RANAM, and RdRP was prepared and dried on Si wafers. Raman spectra were measured using SERS spectra with a Raman spectrometer (SR-303i, Andor Technology) equipped with a 785 nm laser module I0785SR0100B 1 (Innovative Photonic Solutions Inc.).

10. Fabrication of RdRP Detection Prototype Kit

TinkerCad was used to design the prototype kit scaffold. The kit body (1.0 cm×1.2 cm×2.5 cm) is designed to have two holes for loading RANAM and the reaction mixture. The scaffold was printed using a MultiJet 3D printer (3D Systems, ProJet 3510 HD) using a biocompatible UV curable resin (3D Systems, Visijet M3 crystal).

The RANAM was inserted into the control (C) line and test (T) line holes of the detection kit. RdRP positive samples were placed on the T line with 10 μl precursor solution containing 100 pM RdRP. RdRP negative samples were placed on the T line with the same precursor solution without RdRP. The detection kit was incubated at 30° C. for 2 hours. After transfer by RdRP, the TMB solution was added to the C and T lines and the membrane was incubated at room temperature for 5 minutes.

The responsive prototype kit was imaged using a digital camera and filtered with a selective color spot function (Samsung, Galaxy S21) to represent the entire process as color imaging. The RGB digital image was processed into an HSB image using ImageJ software, and the tonal image was represented as pseudocolor. For colorimetric analysis, we used ImageJ software to analyze the saturation intensity of C or T lines based on color images.

### Embodiment 1: Fabrication and Characterization of NA Membranes, Au-NA Membranes, and RANAMs

RANAM's detection of RdRP requires i) a nucleic acid (NA) membrane for RNA polymerase-mediated amplification, and ii) an Au component for colorimetric signal amplification.

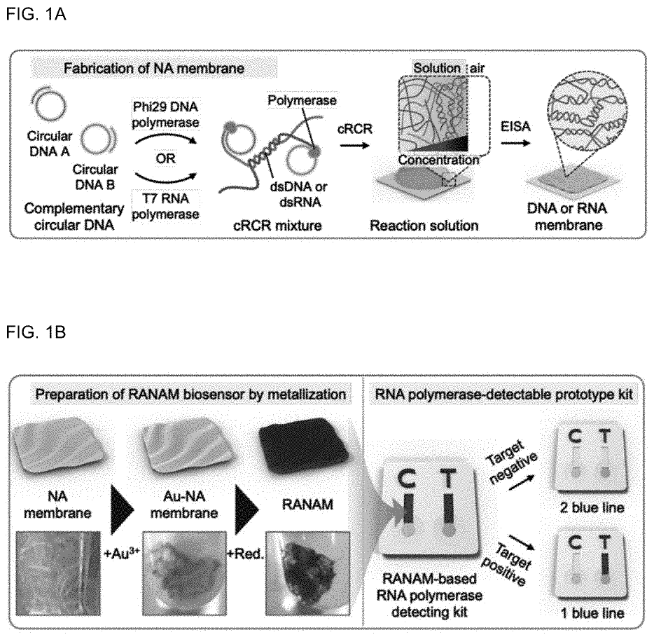

The nucleic acid (NA) membrane scaffold consisted of multiple repeated NA strands generated by cRCR. According to FIG. 1A, cRCA was performed with two complementary circular DNA and phi 29 DNA polymerase to synthesize a DNA membrane, and evaporation-induced self-assembly (EISA) was performed to produce a two-dimensional macroscopic DNA membrane in which DNA strands were highly entangled and concentrated. Similarly, RCTs were performed with two complementary circular DNA strands and T7 RNA polymerase to produce RNA membranes.

According to FIG. 1B, the NA membrane provides many binding sites for the Au cation, and the metallization of the NA membrane induces the enrichment of Au³⁺to produce the Au-NA membrane. Reducing the Au-NA membrane induces Au³⁺ aggregation to induce the growth of Au nanoparticles (AuNPs) inside the Au-NA membrane and expose the Au-covered NA strands. The exposed NA strands can serve as a template for RNA transcriptome generation by RdRP, and the resulting RNA transcripts form shields on the RANAM surface. When RNA transcripts are produced on the surface of RANAM, they are oxidized by Au, blocking the access of TMB, which can show a blue color.

Finally, an RNA-based RANAM biosensor prototype kit was designed to detect viral RdRP. Simply observing the blue lines of the test kit with the naked eye or color imaging using a smartphone can quickly confirm the presence of RdRP.

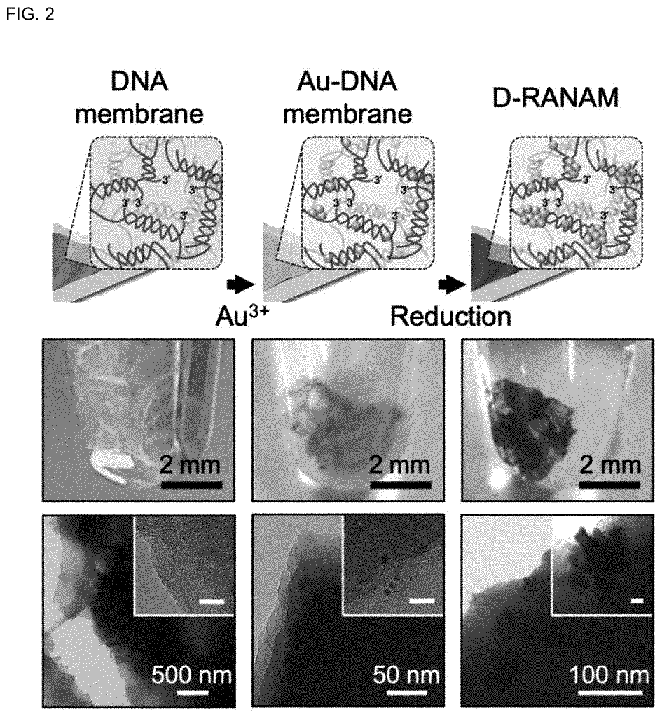

According to FIG. 2, millimeter-sized DNA membranes were successfully synthesized through self-assembly of DNA amplicons extended with circular DNA and phi 29 DNA polymerase. The colorless DNA membrane (NA) contracted and showed yellow color following metallization by gold, indicating high adsorption of gold ions on DNA strands. The Au³⁺ binding DNA membrane (Au-NA) turned into a dark green D-RNAM after the reduction reaction. Transmission electron microscopy (TEM) confirmed that D-RANAM was densely filled with DNA strands bound to gold nanoparticles produced by metallization by gold. Gold ions were agglomerated by reduction and grew into large gold nanoparticles (AuNPs) up to 100 nm in diameter, forming gold nanoclusters exhibiting high electron density.

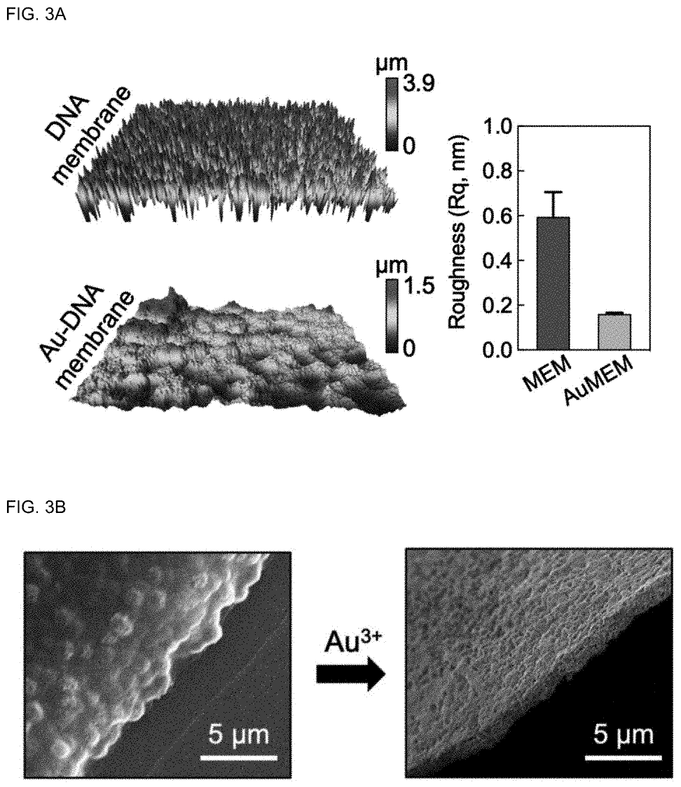

According to the results of contact surface profiler-based analysis of FIG. 3A and FIG. 3B, the rough surface of the DNA membrane is mitigated by metallization by gold. The DNA membrane exhibited a sponge-like rough surface, but the Au-DNA membrane exhibited a thin, flat porous surface after Au metallization. The gold cation progressively coated the DNA membrane to induce morphological flattening of the surface. In addition, the gold cation reduced the repulsive force between the DNA strands representing the anion, leading to the contraction of the DNA membrane. Z-height images observed using AFM showed that the electrostatic and coordination interaction-mediated shrinkage between the Au cation and the membrane reduced the membrane thickness by a factor of two. Despite the morphological changes, the nucleic acid membrane showed such rigidity and stability that the dissolution of the DNA strand did not appear during the gold ion treatment.

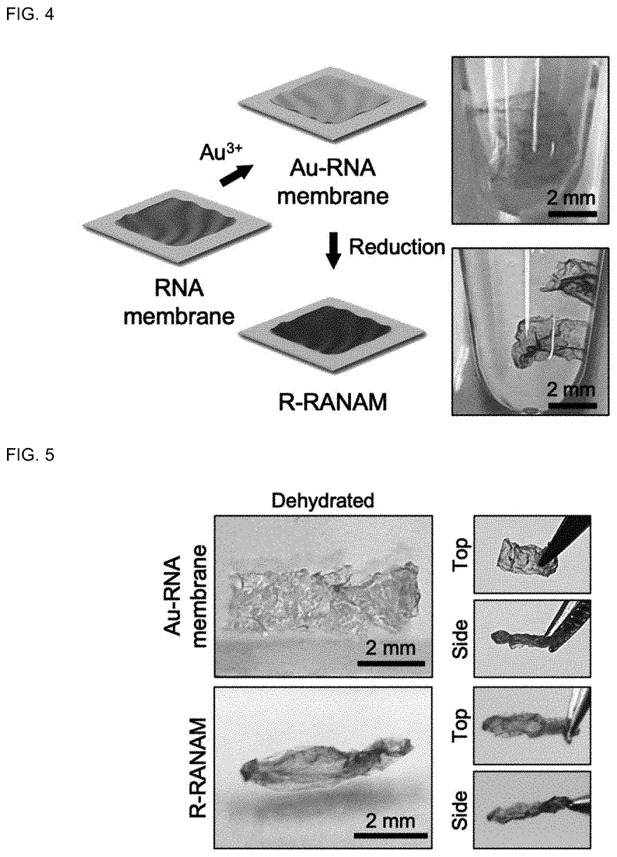

According to FIG. 4, RNA membranes, like DNA membranes, produced dark green RNA-based RANAMs by metallization and reduction by gold.

According to FIG. 5, the Au-RNA membrane and R-RANAM exhibited sufficiently strong mechanical properties.

According to the above experimental results, the nucleic acid membrane can efficiently adsorb gold cations, and both DNA and RNA form a film-like very stable structure by metallization and reduction by gold and have excellent preservation stability. In summary, gold ions bind to nucleic acids, and during the reduction reaction, gold ions act as nanoseeds to form nanoclusters, and the NA strand was shown to be deshielded after the reduction reaction.

### Embodiment 2: Inhibition of RNA Transcription and Gold-Mediated Colorimetric Signal Amplification Using D-RANAM and T7 RNA Polymerase

Nucleic acid membranes embedded with gold ions can undergo gold-mediated redox reactions.

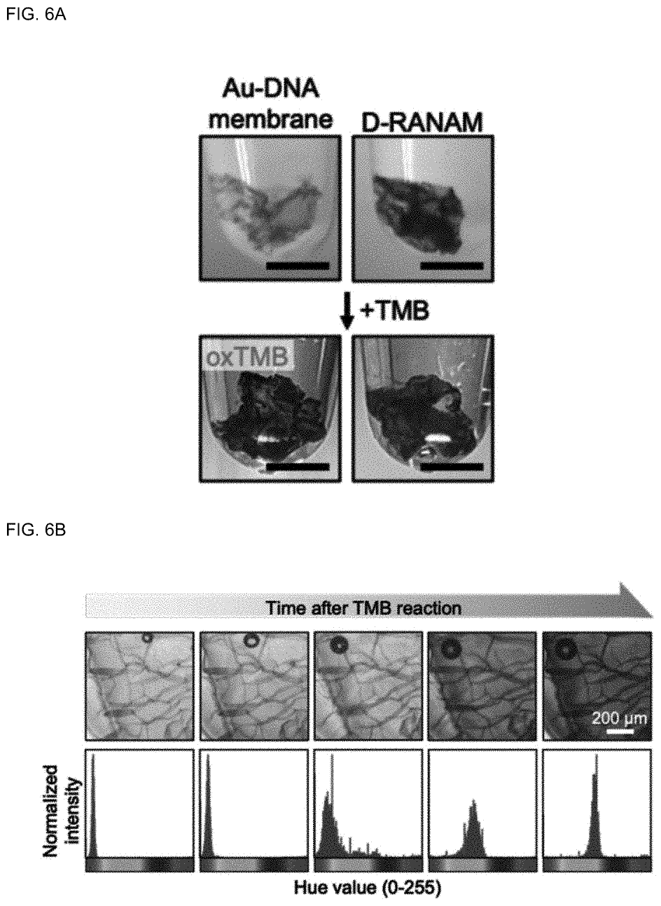

According to FIG. 6A and FIG. 6B, both the Au-DNA membrane and D-RNAM were discolored blue upon contact with TMB. TMB reacted quickly and continuously with the gold component of the membrane, resulting in a gradual color change from yellow to blue, which could be clearly observed with the naked eye.

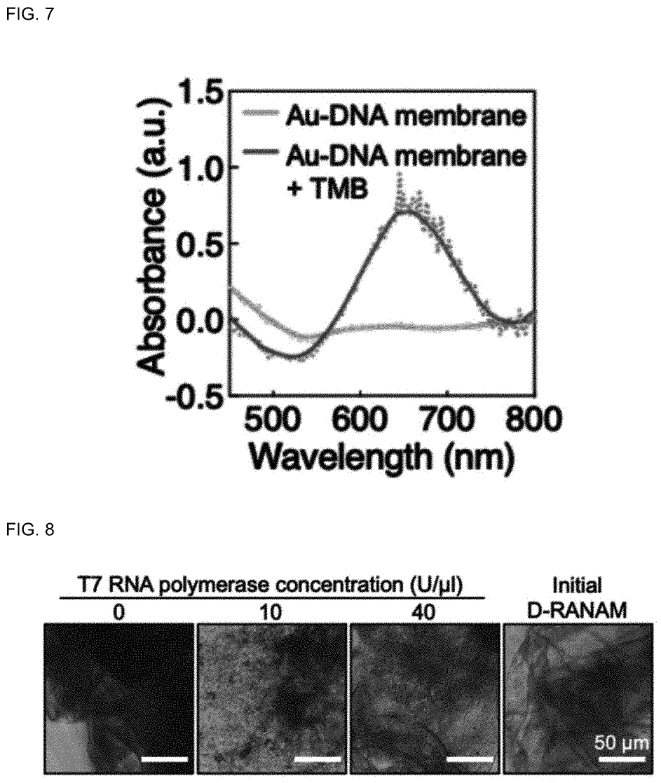

According to FIG. 7, the Au-DNA membrane showed a significant increase in absorbance at about 670 nm upon contact with TMB and a characteristic peak of oxidized TMB (oxTMB). TMB was converted to oxTMB by Au³⁺ and AuNP embedded in the membrane and showed high deposition on the Au-DNA membrane. RNA transcription for RANAM inhibited the degree of redox reaction between RANAM and TMB.

According to FIG. 8, D-RANAM served as a template for RNA transcription upon contact with T7 RNA polymerase and was coated with transcribed RNA. D-RANAM in contact with RNA polymerase significantly reduced the redox reaction between D-RANAM and TMB, thus inhibiting the change to blue color. The result means that when D-RANAM and RNA polymerase come into contact, the RNA strand is amplified on the membrane surface, thereby blocking the reaction between TMB and the gold ion.

### Embodiment 3: Virus RdRP Detection Using RANAM Biosensor

Based on the above experimental results, an R-RANAM-based RNA virus detection sensor targeting the virus RdRP (RNA dependent RNA Polymerase) was fabricated. Since R-RNAM provides a plurality of 3′ terminal sites, it is beneficial for priming RNA transcription by RdRP in COVID-19.

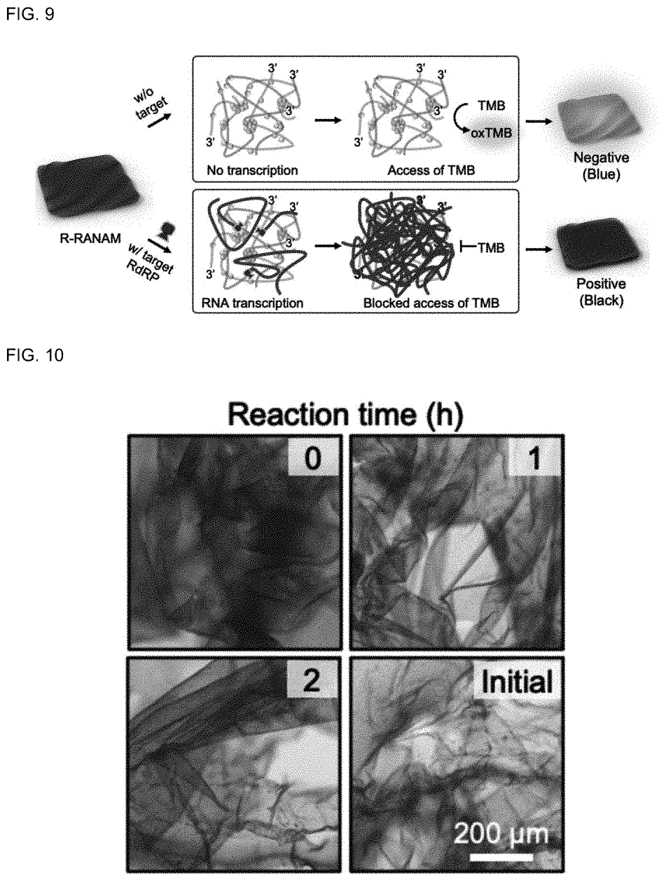

According to FIG. 9, when R-RANAM and RdRP contacted, the newly generated RNA shielded R-RANAM and blocked the redox reaction between the Au component and the TMB substrate, resulting in a decrease in the blue coloration of R-RANAM.

According to FIG. 10, the time required for blue coloration of R-RANAM increased with transcription time.

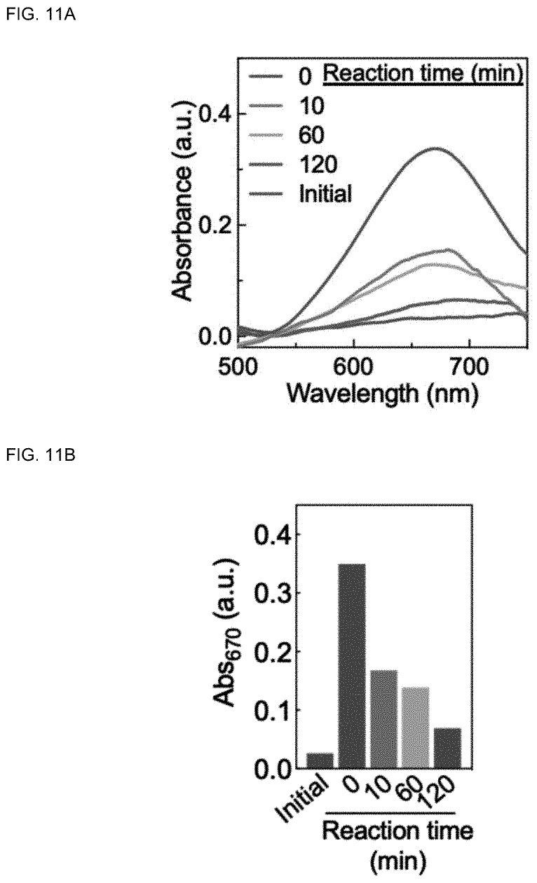

According to FIG. 11A, R-RANAM has a gradual decrease in the 670 nm peak height due to oxidation TMB when the transcription time by RdRP increases.

According to FIG. 11B, even a transcription time of only 10 minutes reduced absorbance by half. This may be due to an RdRP-mediated cascade reaction in which the 3′ end of the newly generated RNA acts as a template again. R-RANAM with a transcription time of 2 hours was significantly reduced in a change in color to blue to the extent that it was similar in color to R-RANAM without transcription. This means that Au³⁺ and AuNPs embedded in the membrane are efficiently shielded by RNA newly generated by RdRP.

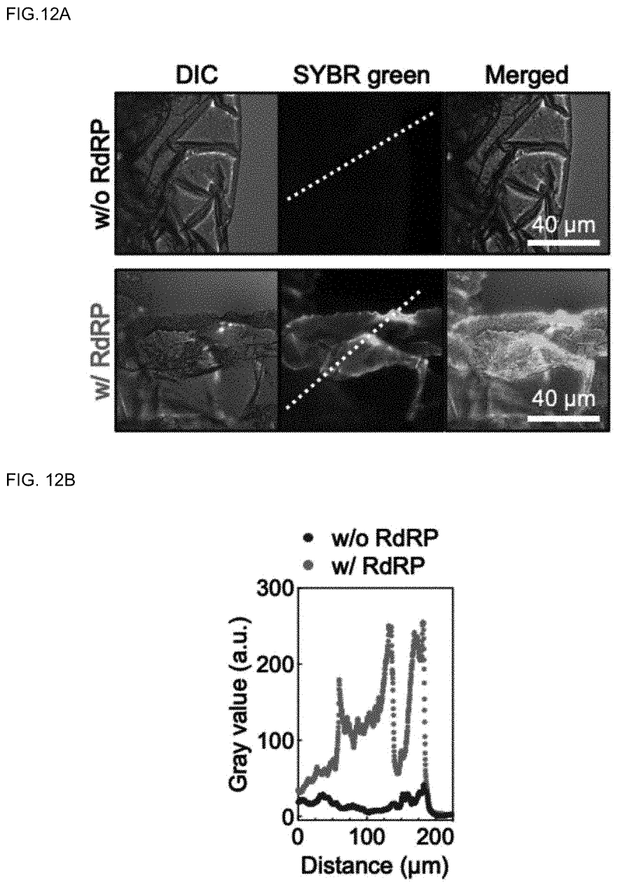

According to FIG. 12A, in order to demonstrate that RNA is amplified when R-RANAM is contacted with RdRP, the RNA of R-RANAM was stained using SYBR green I, an NA insertion dye.

According to FIG. 12B, R-RANAM stained with SYBR green I showed very high fluorescence signal amplification in reaction with RdRP. This indicates that RdRP successfully replicated RNA with an RNA membrane as a template.

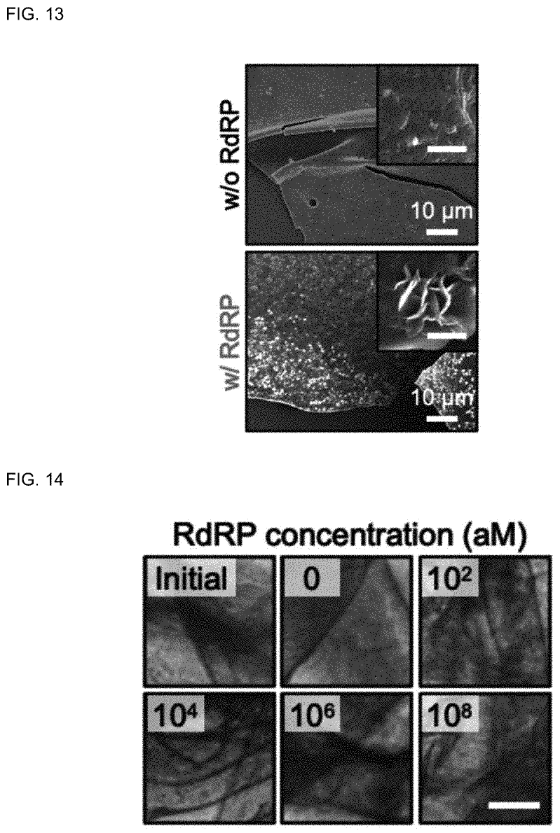

According to FIG. 13, it was confirmed that R-RANAM reacted with RdRP to form RNA strands, and the flower-like RNA structure expanded to cover the R-RANAM surface. The RNA structure generated on the surface of RANAM by RdRP is referred to as an RNA bump. In summary, the RNA bump structure prevents Au³⁺ and AuNP from contacting TMB, thereby inhibiting the color change to blue.

However, the Au-RNA membrane that did not undergo reduction did not inhibit the TMB redox reaction even after incubation with RdRP. These results imply that 3′-terminal exposure of RNA by reduction is important for RNA transcript amplification by RdRP.

In addition, the R-RANAM scaffold exhibited a characteristic Raman spectrum of RNA compared to simple RNA membranes due to the embedded AuNP. The RNA Raman peak of R-RANAM was significantly enhanced by RNA strand amplification of the R-RANAM surface by RdRP. These results mean that the R-RANAM platform can be applied as a label-free Raman based biosensor that does not require labels or probes to detect RdRP.

### Embodiment 4: Concentration-Dependent RdRP Detection Using Design-Free R-RANAM Biosensor

To investigate the RdRP detection limits of the R-RANAM biosensor, the membrane was treated with various concentrations of RdRP.

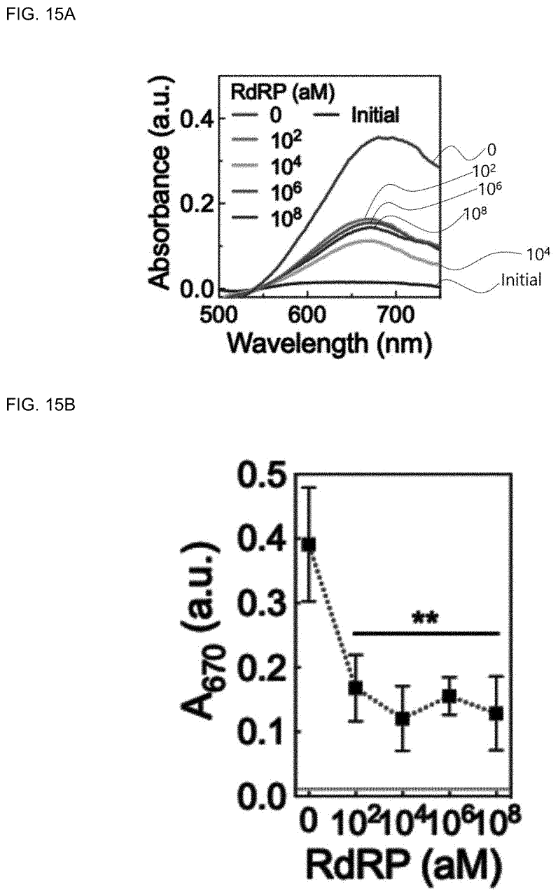

According to the digital image of FIG. 14, the blue pigmentation gradually decreased as the RdRP concentration increased from 10² to 10⁸ aM. This suggests that RNA bumps tightly entangled on the membrane surface can successfully shield the Au embedded in the membrane.

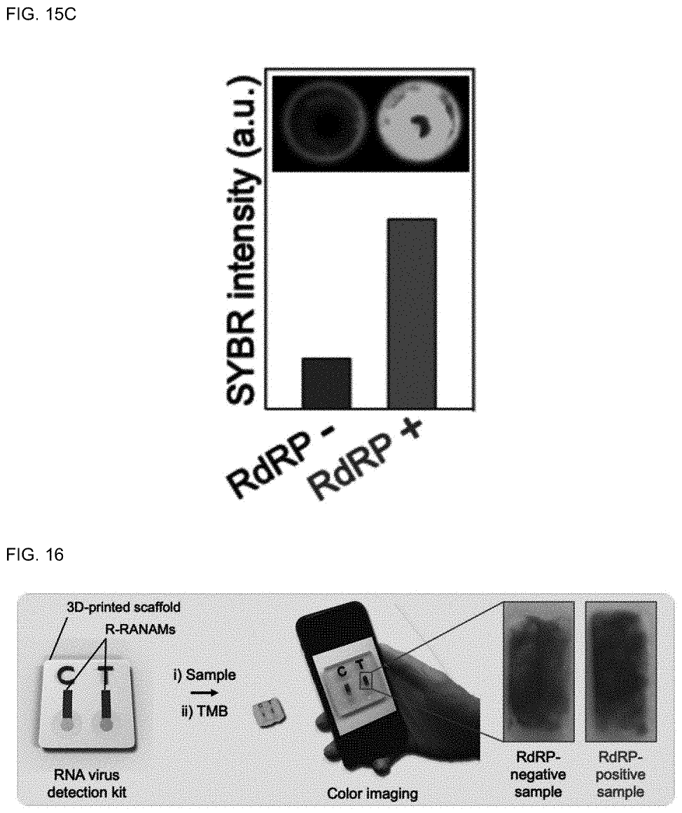

According to FIG. 15A and FIG. 15B, the R-RANAM treated with RdRP significantly reduced the absorbance of 670 nm compared to R-RANAM without RdRP. R-RANAM showed very high sensitivity by processing only 100 aM of RdRP with significantly less discoloration than the control group. An RdRP of 100 aM corresponds to an RdRP of about 600 copies. This means that RANAM-based virus detection systems have a sensitivity high enough to detect RdRPs as low as about 600 copies. In addition, short-term culture with RdRP was also tested and concentration-dependent signal reduction was identified. This indicates that the longer the incubation time, the greater the sensitivity even at low RdRP concentrations. The sensitivity of R-RANAM was similar to that of RNA gene specific detection systems. As a result of the experiment staining R-RANAM with SYBR green I dye, the green fluorescence of RdRP-treated R-RANAM was much higher than that of untreated R-RANAM, indicating that RNA replication was efficiently achieved. This suggests that R-RANAM can also be utilized for RdRP detection using fluorescence analysis instruments.

In addition, the detection sensitivity of the R-RANAM biosensor was independent of the RNA sequence of the RNA membrane. Taken together, R-RANAM can provide a design-free biosensor that can be widely applied to the detection of various RNA viruses.

### Embodiment 5: RdRP Detection-Based Prototype Kit for Virus Detection

The R-RANAM biosensor has the advantage of being able to detect the presence of the virus with the naked eye. Based on this, an easy-to-use virus diagnostic prototype kit was devised. The kit was built using a 3D printed scaffold and includes a control (C) and test (T) line with two R-RANAMs built-in.

According to FIG. 16, the kit placed the sample and the reaction mixture on the T line and the TMB on two lines, C and T, so that the presence of viral RdRP could be visually confirmed based on the number of blue lines.

In addition, color imaging from commonly used smartphone cameras was used to precisely recognize and distinguish the colors of diagnostic kits.

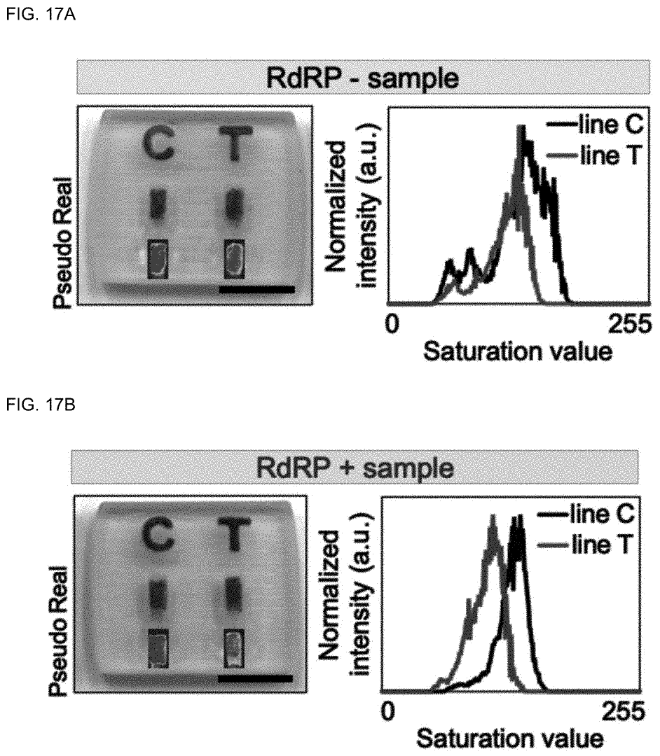

According to FIG. 17A, the RdRP negative sample was marked with two blue lines on both the C and T lines. This indicates that the Au of the membrane reacted successfully with TMB.

On the other hand, according to FIG. 17B, the RdRP positive sample processing kit showed a blue line at C but no blue at T. Visualizing the results through the pseudo-color effect or quantifying the saturation value made it easier to distinguish the colors of the C and T lines.

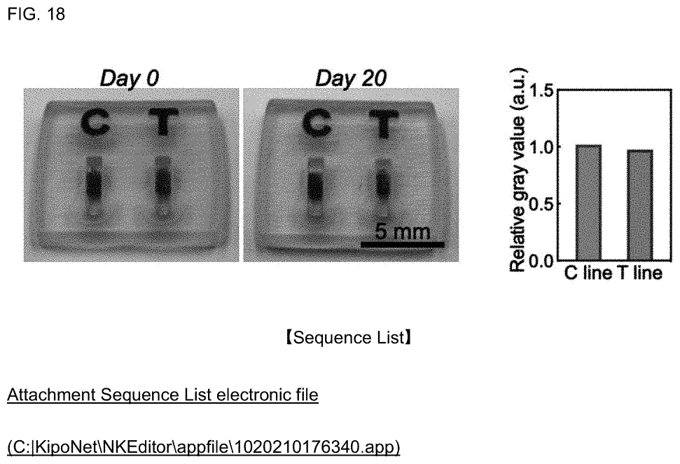

On the other hand, according to FIG. 18, R-RANAM can maintain gray value even when stored at room temperature for more than 20 days, so it is advantageous for commercialization because it has sufficient stability to store with a Pre-loaded Kit.

In conclusion, the R-RANAM biosensor of the present disclosure can independently detect viral sequences, can be applied to detect mutations of SARS-CoV-2, and has great potential as a smartphone auxiliary virus detection kit capable of self-diagnosis.

What is claimed is:

1. An RNA polymerase-activated nucleic acid membrane comprising:
a plurality of nucleic acid strands partially complementary; and
a gold component bound to the nucleic acid strand;
wherein the nucleic acid strand comprises an exposed end that reacts with RNA polymerase.

2. The RNA polymerase-activated nucleic acid membrane of claim 1, wherein the nucleic acid is DNA or RNA.

3. The RNA polymerase-activated nucleic acid membrane of claim 1, wherein the RNA polymerase is T7 RNA polymerase or RNA-dependent RNA polymerase (RdRP).

4. The RNA polymerase-activated nucleic acid membrane of claim 1, wherein the gold component is a gold ion, gold nanoparticles, or a combination thereof.

5. The RNA polymerase-activated nucleic acid membrane of claim 1, wherein a structure consisting of RNA transferred on the surface is formed when the nucleic acid membrane reacts with RNA polymerase.

6. A Biosensor for RNA polymerase detection, comprising:
the RNA polymerase-activated nucleic acid membrane of claim 1.

7. A Kit for virus detection containing RNA polymerase, comprising:
the biosensor of claim 6 and a dye that changes color by gold and redox reaction.

8. A method for RNA polymerase-activated nucleic acid membrane manufacturing, the method comprising:
preparing a nucleic acid membrane;
mixing the nucleic acid membrane with the gold ion to prepare a nucleic acid membrane in which the gold ion is bound; and
reducing the nucleic acid membrane to which the gold ion binds.
